(19) United States
(12) Patent Application Publication
(10) Pub. No.: US 2022/0273615 A1
(43) Pub. Date: Sep. 1, 2022

(54) METHODS OF TREATING POMPE DISEASE

(71) Applicant: Academia Sinica, Taipei (TW)

(72) Inventors: Wei-Chieh CHENG, Taipei (TW); Huang-Yi LI, Taipei City (TW); Wuh-Liang HWU, Taipei (TW); Yin-Hsiu CHIEN, Taipei (TW); Ni-Chung LEE, Taipei (TW)

(73) Assignee: Academia Sinica, Taipei (TW)

(21) Appl. No.: 17/630,929

(22) PCT Filed: Sep. 30, 2020
(86) PCT No.: PCT/US20/53352

§ 371 Date: Jan. 28, 2022

Related U.S. Application Data
(60) Provisional application No. 62/910,552, filed on Oct. 4, 2019.

(51) Int. Cl. A61K 31/40; A61K 38/47; A61P 3/08

(52) U.S. Cl. CPC A61K 31/40; A61K 38/47; C12Y 302/0102; A61P 3/08

(57) ABSTRACT

Disclosed herein are novel uses of ADMDP stereoisomers or their derivatives for the manufacture of a medicament for treating Pompe disease. Accordingly, the present disclosure provides a method of treating Pompe disease in a subject. The method includes the step of, administering to the subject a therapeutically effective amount of a compound of formula (I), or a salt, an ester or a solvate thereof, wherein R₁ and R₂ are independently H or alkyl optionally substituted by —NH₂ or —OH, so as to ameliorate, alleviate mitigate and/or prevent symptoms associated with the Pompe disease. According to certain embodiments of the present disclosure, the compound of formula (I) may serve a stabilizer of α-glucosidase via preventing its denaturalization of deactivation.

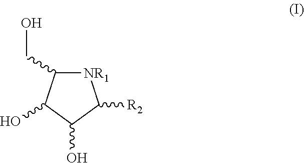

### CROSS-REFERENCE TO RELATED APPLICATIONS

This application relates to and claims the benefit of U.S. Provisional Application No. 62/910,552, filed Oct. 4, 2019; the content of the application is incorporated herein by reference in its entirety.

### BACKGROUND OF THE INVENTION

### 1. Field of the Invention

The present disclosure in general relates to the field of disease treatment. More particularly, the present disclosure relates to the uses of polyhydroxylated pyrrolidines for the manufacture of a medicament for treating Pompe disease.

### 2. Description of Related Art

Pompe disease (PD), also known as glycogen storage disease type II, is a lysosomal storage disease caused by mutations of α-glucosidase (GAA) encoding gene. GAA plays a critical role in hydrolyzing lysosomal glycogen, and deficiency of α-glucosidase results in abnormal glycogen accumulation in the lysosomes of heart, muscle, and liver. Pompe disease displays a board phenotypic spectrum ranges from severe infantile-onset form to mild later-onset form, and Pompe patients mostly suffer from the progressive muscle hypotonia and respiratory failure. The estimated incidence of Pompe disease is about 1 in 40,000 live births.

Enzyme replacement therapy (ERT) is the first Food and Drug Administration (FDA) approved treatment for Pompe patients in 2006. Recombinant human α-glucosidase (rh-α-glu, rhGAA) is injected into the patient, after which it would be transported into cells through endocytosis, eventually reducing accumulated substrates thereby reducing pathological conditions and delaying the need for invasive ventilator support in infantile Pompe patients. However, rhGAA is unstable at neutral pH and body temperature, and accordingly, high dose of rhGAA (10 times more than other diseases) is required to achieve the therapeutic effect. Considering the fact that the production and purification of GAA are expensive procedures, and frequently repeated administration of GAA often elicits immune response that adversely affects tolerability and therapeutic efficacy, there exists in the related art a need for a method for improving the pharmacological property of GAA so as to enhance its efficacy in treating Pompe disease.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

The present disclosure is based on unexpected discovery that certain polyhydroxylated pyrrolidines are potent GAA stabilizers, which protect GAA from protein denaturalization and/or deactivation. Thus, these polyhydroxylated pyrrolidines may serve as molecular stabilizers of GAA (including rhGAA or mutant GAA), and therefore are useful for the development of medicaments for the treatment or prophylaxis of Pompe disease.

Accordingly, the present disclosure is directed to a method of treating Pompe disease in a subject by use of a polyhydroxylated pyrrolidine. The method comprises administering to the subject a first therapeutic effective amount of a compound of formula (I), or a salt, an ester or a solvate thereof,

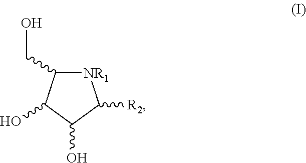

wherein R₁and R₂are independently H or alkyl optionally substituted by —NH₂ or —OH; so as to ameliorate, alleviate mitigate and/or prevent symptoms associated with the Pompe disease.

According to preferred embodiments of the present disclosure, R₁ is H, and R₂ is H or methyl optionally substituted by —NH₂ or —OH; or R₁ is methyl, and R₂ is H.

According to some preferred embodiments, the compound of formula (I) is selected from the group consisting of,

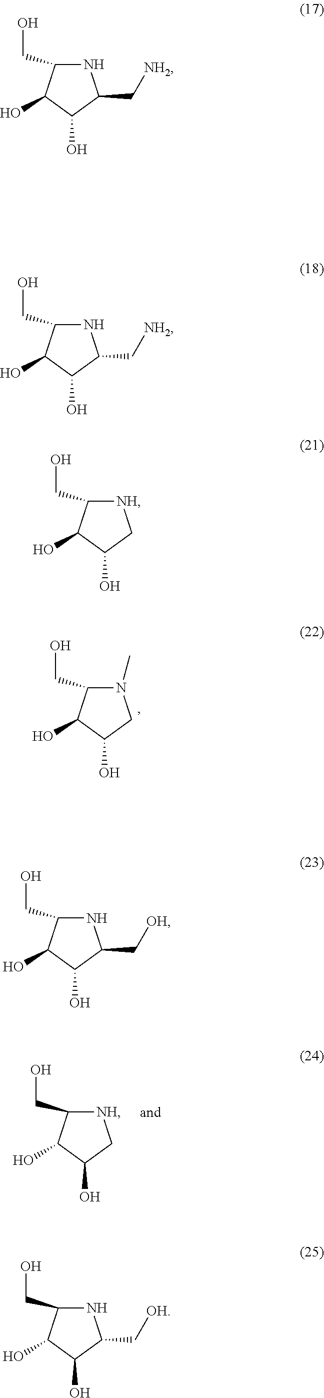

In certain preferred embodiments, the compound of formula (I) is selected from the group consisting of,

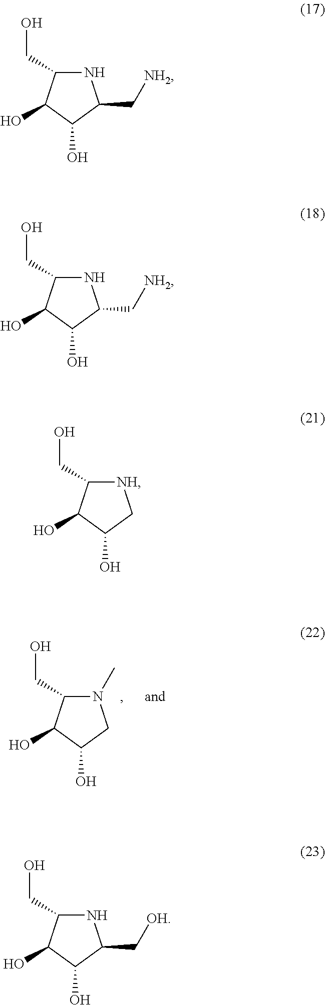

According to one preferred embodiment, the compound of formula (I) is

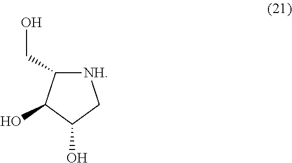

According to another preferred embodiment, the compound of formula (I) is

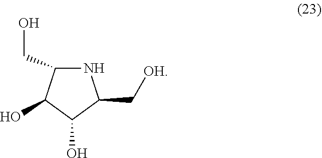 [SMILES: OC[C@@H]1N[C@@H](CO)[C@H](O)[C@H]1O]

According to some embodiments of the present disclosure, the compound of formula (I) is administered to the subject in an amount of about 0.01 mg/Kg to 10 g/Kg. Preferably, the compound of formula (I) is administered to the subject in an amount of about 0.1-1,000 mg/Kg. More preferably, the compound of formula (I) is administered to the subject in an amount of about 1-100 mg/Kg.

Optionally, the present method further comprises administering to the subject a second therapeutically effective amount of GAA, prior to, concurrently with, or after the administration of the compound of formula (I), or the salt, the ester or the solvate thereof.

The subject is a mammal; preferably, a human.

Many of the attendant features and advantages of the present disclosure will becomes better understood with reference to the following detailed description considered in connection with the accompanying drawings.

### BRIEF DESCRIPTION OF THE DRAWINGS

The present description will be better understood from the following detailed description read in light of the accompanying drawings, where:

FIG. 1 is a line chart depicting the results of thermal shift assay according to Example 1 of the present disclosure.

FIGS. 2A-2D are line charts respectively depicting the results of thermal shift assay according to Example 2 of the present disclosure. FIG. 2A: the misfolding percentages of recombinant human α-glucosidase (rhGAA) incubated with specified compounds in a phosphate buffer (pH 7.0) at 48° C. for indicated period of time. FIG. 2B: the relative activity (%) of rhGAA incubated in DMEM medium at 37° C. for 15, 30, 45 or 60 minutes. FIGS. 2C and 2D: the relative activity (%) of rhGAA incubated with compound 21 (FIG. 2C) or compound 23 (FIG. 2D) in a phosphate buffer (pH 7.0) at 48° C. for 10, 20 or 30 minutes. NT: no treatment.

FIGS. 3A-3E are histograms and line chart respectively depicting the enzyme activities in cells according to Example 3 of the present disclosure. FIGS. 3A and 3B: the activity of GAA in D645E fibroblasts, which were treated with rhGAA (0.05, 0.5, or 5 μM) in the absence (NT) or presence of compound 21 or 23 (50 μM) for 24 hours. FIG. 3C: the relative activity of GAA in D645E fibroblasts, which were treated with rhGAA (0.5 μM) in the absence (NT) or presence of specified compound (compound 21 or NB-DNJ). FIG. 3D: The glycogen content in D645E fibroblasts, which were treated with rhGAA (0.5 μM) in the absence or presence of compound 21 (0.1, 1 or 10 μM), compared to NT (no treatment). FIG. 3E: the relative activity of GAA in M519V fibroblasts, which were treated with compound 21 at specified concentrations to see the chaperoning effect. The data points were depicted as a mean±SDM of 3 wells tested in parallel from one representative of three independent experiments. NB-DNJ: N-butyl-deoxynojirimycin, serving as a positive control in the present invention. NT: no treatment. Enzyme: rhGAA treatment.

FIG. 4A and 4B are histograms respectively depicting the activity of GAA (FIG. 4A) and the glycogen content (FIG. 4B) in the hearts of mice treated with specified treatments according to Example 4 of the present disclosure. WT control: wild-type mice. Untreated: Pompe mice without treatment. ERT: Pompe mice treated with enzyme replacement therapy. ERT+NB-DNJ: Pompe mice treated with enzyme replacement therapy and NB-DNJ. ERT+compound 21: Pompe mice treated with enzyme replacement therapy and compound 21.

### DETAILED DESCRIPTION OF THE INVENTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### I. Definition

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about”. Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

As used herein, the term “substituted,” when used to describe a chemical structure or moiety, refers to a derivative of that structure or moiety, wherein one or more of its hydrogen atoms is/are substituted with one or more substituent(s), for example, being substituted with one or more —NH₂ and/or —OH. Unless otherwise indicated, a “substituted” structure or moiety has a substituent at one or more substitutable positions of the structure or moiety, and when more than one position in any given structure or moiety is substituted, the substituent is either the same or different at each position.

As used herein, the term “optionally substituted” in connection with a chemical structure or moiety refers to the structure or moiety that is unsubstituted or substituted.

“Alkyl” refers to a radical of a straight-chain or branched saturated hydrocarbon group having from 1 to 20 carbon atoms (“C₁₋₂₀ alkyl”). In some embodiments, an alkyl group has 1 to 10 (“C₁₋₁₀ alkyl”), 1 to 9 (“C₁₋₉ alkyl”), 1 to 8 (“C₁₋₈ alkyl”), 1 to 7 (“C₁₋₇ alkyl”), 1 to 6 (“C₁₋₆ alkyl”), 1 to 5 (“C₁₋₅ alkyl”), 1 to 4 (“C₁₋₄ alkyl”), 1 to 3 (“C₁₋₃ alkyl”), 1 to 2 (“C₁₋₂ alkyl”) carbon atoms. The alkyl group may also refer to 1 carbon atom (“C₁ alkyl”).

The term “solvate” herein refers to a complex formed by the interaction of a compound (such as the compound of formula (I) of this invention) with surrounding solvent molecules, such as water, alcohol and other polar organic solvents. Non-limiting examples of alcohol include methanol, ethanol, n-propanol, isopropanol, n-butanol, isobutanol, and t-butanol. Examples of alcohol also include polymerized alcohols, such as polyalkylene glycols (e.g., polyethylene glycol, and polypropylene glycol). The best-known and preferred solvent is typically water, and solvate compounds formed by solvation with water are termed hydrates.

It should also be noted that if the stereochemistry of a structure or a portion of a structure is not indicated with, for example, bold or dashed lines, the structure or the portion of the structure is to be interpreted as encompassing all stereoisomers of it. Similarly, names of compounds having one or more chiral centers that do not specify the stereochemistry of those centers encompass pure stereoisomers and mixtures thereof. Moreover, any atom shown in a drawing with unsatisfied valences is assumed to be attached to enough hydrogen atoms to satisfy the valences.

As used herein, the term “treatment” includes preventative (e.g., prophylactic), curative or palliative treatment of a disease in a mammal, particularly human; and includes: (1) preventative (e.g., prophylactic), curative or palliative treatment of a disease or condition (e.g., Pompe disease) from occurring in an individual who may be pre-disposed to the disease but has not yet been diagnosed as having it; (2) inhibiting a disease (e.g., by arresting its development and/or process); or (3) relieving a disease (e.g., reducing symptoms associated with the disease).

The term “administered,” “administering” or “administration” are used interchangeably herein to refer a mode of delivery, including, without limitation, orally, topically, mucosally, transdermally and parenterally (such as intravenously, intra-arterially, intramuscularly, and subcutaneously) administering an agent (e.g., the compound of formula (I)) of the present invention.

Unless otherwise indicated, a “therapeutically effective amount” of a compound is an amount sufficient to provide a therapeutic benefit in the treatment or management of a disease or condition, or to delay or minimize one or more symptoms associated with the disease or condition. A therapeutically effective amount of a compound is an amount of therapeutic agent, alone or in combination with other therapies, which provides a therapeutic benefit in the treatment or management of the disease or condition. The term “effective amount” can encompass an amount that improves overall therapy, reduces or avoids symptoms or causes of a disease or condition, or enhances the therapeutic efficacy of another therapeutic agent. Persons having ordinary skills could calculate the human equivalent dose (HED) for the agent (such as the compound of formula (I) of the present invention) based on the doses determined from animal models. For example, one may follow the guidance for industry published by US Food and Drug Administration (FDA) entitled “Estimating the Maximum Safe Starting Dose in Initial Clinical Trials for Therapeutics in Adult Healthy Volunteers” in estimating a maximum safe dosage for use in human subjects.

The term “subject” or “patient” refers to a mammal including the human species that is treatable with the compound of formula (I) and/or method of the present invention. The term “subject” is intended to refer to both the male and female gender unless one gender is specifically indicated.

### II. Description of The Invention

Compounds useful in this invention are stereoisomers of 1-aminodeoxy-DMDP (2,5-dideoxy-2,5-imino-d-mannitol, DMDP) (ADMDP), and derivatives thereof. The chemical structure of ADMDP comprises at least 4 asymmetric carbon atoms (i.e., chiral centers); thus, ADMDP encompasses at least 16 stereoisomers. Inventors of the present invention unexpectedly discovered that two of these ADMDP stereoisomers (i.e., compounds 17 and 18), and their derivatives (i.e., compounds 21-25) are useful in stabilizing the activity of GAA, and accordingly, may be used as potential lead compounds for the development of medicaments for treating Pompe disease.

The present disclosure thus provides a method of treating Pompe disease by use of specified ADMDP stereoisomer or the derivative thereof. Specifically, the method for treating Pompe disease in a subject comprises administering to the subject a therapeutically effective amount of a compound of formula (I), or a salt, an ester or a solvate thereof,

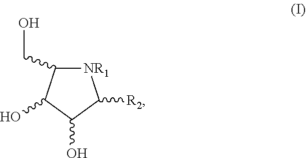

wherein R₁ and R₂ are independently H or alkyl optionally substituted by —NH₂ or —OH.

According to preferred embodiments of the present disclosure, R₁ is H, and R₂ is H or methyl optionally substituted by —NH₂ or —OH; alternatively, R₁ is methyl, and R₂ is H.

Examples of the compound of formula (I) include, but are not limited to,

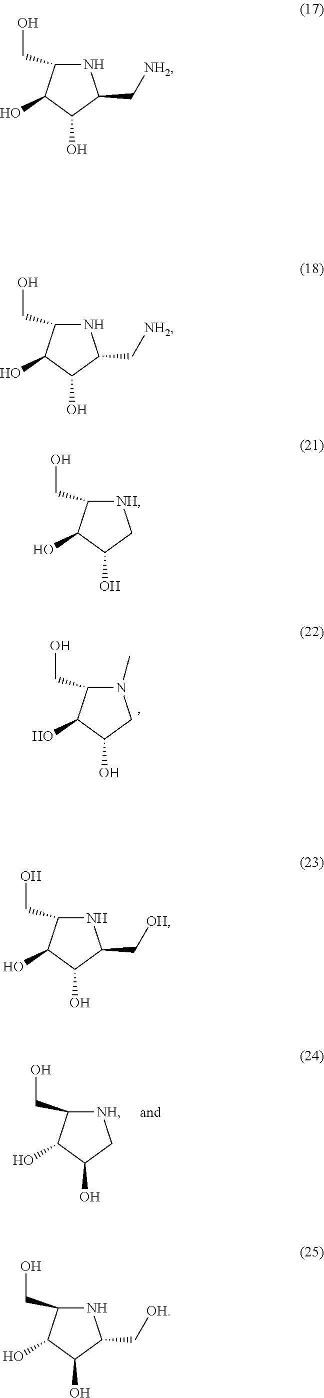

According to certain embodiments, the compound of the present disclosure has the structure of formula (I-1),

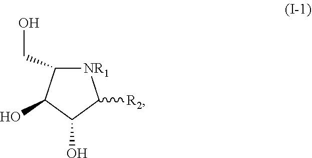

wherein R₁ and R₂ are independently H or alkyl optionally substituted by —NH₂ or —OH.

Preferably, the compound of formula (I-1) is selected from the group consisting of,

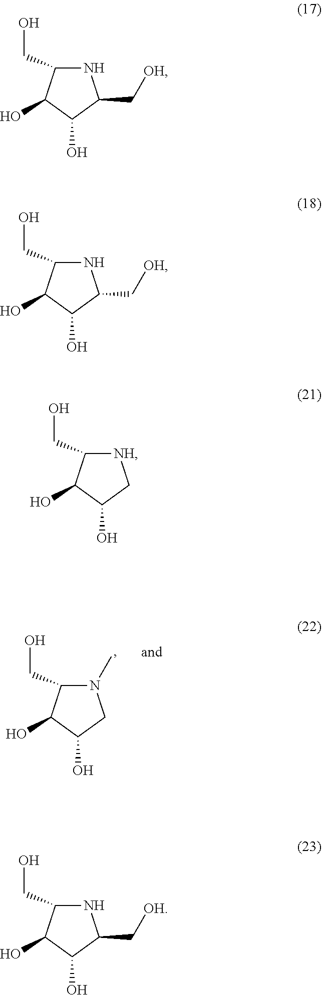

In one preferred embodiment, the compound of formula (I-1) is

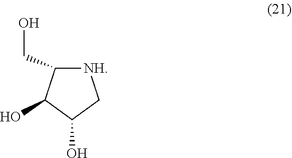

In another preferred embodiment, the compound of formula (I-1) is

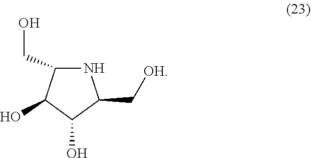 [SMILES: OC[C@@H]1N[C@@H](CO)[C@H](O)[C@H]1O]

According to some embodiments of the present disclosure, the compound of formula (I) is capable of preventing GAA from denaturalization or deactivation thereby stabilizing the activity of GAA. Thus, in some optional embodiments, the method further comprises administering to the subject a therapeutically effective amount of GAA, prior to, concurrently with, or after the administration of the compound of formula (I), or the salt, the ester or the solvate thereof.

According to certain embodiments of the present disclosure, the subject is a mouse. To elicit a therapeutic effect in mice, the compound of formula (I) is administered to the subject in an amount of about 0.1 mg/Kg to 100 g/Kg body weight per dose. Preferably, the compound of formula (I) is administered to the subject in an amount of about 1 mg/Kg to 10 g/Kg body weight per dose. More preferably, the compound of formula (I) is administered to the subject in an amount of about 10 to 1,000 mg/Kg body weight per dose. According to one specific example, 100 mg/Kg or 200 mg/Kg of the compound of formula (I) is sufficient to elicit a therapeutic effect on the subject.

A skilled artisan may readily determine the human equivalent dose (HED) of the compound of formula (I), based on the doses determined from animal studies provided in working examples of the present application. The amount of the compound of formula (I) suitable for use in a human subject may be in the range of 0.01 mg/Kg to 10 g/Kg body weight per dose, such as 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, or 990 mg/Kg per dose; or 1, 1.5, 2, 2.5, 3, 3.5, 4, 4.5, 5, 5.5, 6, 6.5, 7, 7.5, 8, 8.5, 9, 9.5, or 10 g/Kg per dose. Preferably, the amount of the compound of formula (I) suitable for use in a human subject is in the range of 0.1 to 1,000 mg/Kg per dose. More preferably, the amount of the compound of formula (I) suitable for use in a human subject is in the range of 1-100 mg/Kg per dose. In one specific embodiment, the HED of the compound of formula (I) is about 5-20 mg/Kg per dose.

For the purpose of efficiently increasing the activity of GAA, the compound of formula (I) may be administered to the subject one or more times. For example, the compound of formula (I) may be administered once for a full course of treatment. Alternatively, the compound of formula (I) may be administered to the subject once every day, every two days, every three days, every four days, every five days, every six days, every week, every two weeks, every three weeks, every month, every two months, every three months, every fourth months, every five months, or longer period of time (e.g., once per year). According to some examples, the compound of formula (I) is administered to the subject once per week.

The compound of formula (I) may be formulated with a suitable pharmaceutical excipient or carrier, and manufactured into a medicament (e.g., a pharmaceutical composition or formulation). The compound of formula (I) may be present at a level of about 0.1% to 99% by weight, based on the total weight of the medicament. In some embodiments, the compound of formula (I) is present at a level of at least 1% by weight, based on the total weight of the medicament. In certain embodiments, the compound of formula (I) is present at a level of at least 5% by weight, based on the total weight of the medicament. In still other embodiments, the compound of formula (I) is present at a level of at least 10% by weight, based on the total weight of the medicament. In still yet other embodiments, the compound of formula (I) is present at a level of at least 25% by weight, based on the total weight of the medicament.

The medicament may be formulated in single unit dosage form suitable for oral, mucosal (e.g., nasal, sublingual, vaginal, buccal, or rectal), parenteral (e.g., subcutaneous, intravenous, bolus injection, intramuscular, or intra-arterial), or transdermal administration to the subject. Examples of dosage form include, but are not limited to, tablet, caplet, capsule (such as soft elastic gelatin capsule), dispersion, suppository, ointment, cataplasm, paste, powder, dressing, cream, plaster, solution, patch, aerosol (e.g., nasal spray or inhaler), gel, suspension (e.g., aqueous or non-aqueous liquid suspension, oil-in-water emulsion, or water-in-oil emulsion), solutions, and elixir.

The medicament may optionally comprise one or more additives to improve or enhance the taste flavor, absorption and/or performance of the medicament, such as flavoring agent, lubricant, suspending agent, filler, glidant, compression aid, binder, tablet-disintegrating agent, nutritional supplement, anti-oxidant, dispersant, thickener, colorant, an encapsulating material, or a combination thereof.

Depending on desired purposes, the medicament may be administered to the subject via a route selected from the group consisting of oral, enteral, nasal, topical, transmucosal, transdermal, and parenteral administration, in which the parental administration is any of intramuscular, intravenous, intra-arterial, subcutaneous, or intraperitoneal injection.

As could be appreciated, the amount, route of administration, and dosing schedule of the compound of formula (I) or the medicament comprising the same may depend upon factors such as the specific symptoms to be treated, prevented, or managed, and the age, sex and condition of the patient. The roles played by such factors are well known in the art, and may be accommodated by routine experimentation.

The present method can be applied to the subject, alone or in combination with additional therapies that have some beneficial effects on the prevention or treatment of Pompe disease. Depending on the intended/therapeutic purpose, the present method can be applied to the subject before, during, or after the administration of the additional therapies.

The subject treatable with the present method is a mammal, for example, a human, a mouse, a rat, a monkey, a rabbit, a dog, a cat, a sheep, a goat, a horse, or a chimpanzee. Preferably, the subject is a human.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### EXAMPLE

### Materials and Methods

Preparation of Compounds 17-20

Compounds 17-20 were prepared in accordance with the method published previously. See, e.g., Tsou EL et al., 2009, Tetrahedron 2009, 65: 93-100.

Preparation of Compound 21

A solution of compound 19 (121 mg, 0.29 mmol) in MeOH was treated with palladium hydroxide in a hydrogen atmosphere for 24 hours. The reaction mixture was filtered through Celite, concentrated, and purified by CC (column chromatography) to give compound 21 as a yellowish oil (26 mg, 0.19 mmol, 67%). ¹H NMR (600 MHz, D₂O) δ 3.13 (dd, 1H, J=2.4, 12.1 Hz), 3.34-3.38 (m, 2H), 3.69 (dd, 1H, J=7.8, 12.0 Hz), 3.81 (dd, 1H, J=4.2, 12.0 Hz), 3.93 (dd, 1H, J=3.6, 3.7 Hz), 4.17-4.21 (m, 1H); ¹³C NMR (150 MHz, D₂O) δ 76.5, 75.1, 66.2, 59.7, 50.1; HRMS calcd for [C₅H₁₁NO₃+H]⁺ 134.0812, found 134.0812.

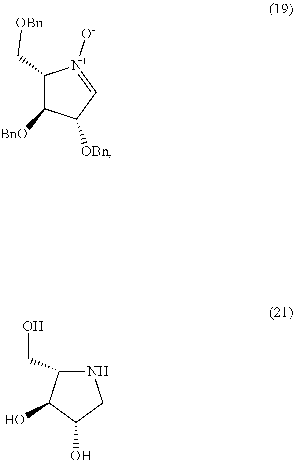 [SMILES: [O-][N+]1=C[C@H](OCc2ccccc2)[C@@H](OCc2ccccc2)[C@@H]1COCc1ccccc1 ; OC[C@@H]1NC[C@H](O)[C@H]1O]

Preparation of Compound 22

A solution of compound 21 (20 mg, 0.15 mmol) in MeOH was treated with palladium hydroxide and formaldehyde (100 μL, 1.5 mmol) in a hydrogen atmosphere for 24 hours. The reaction mixture was filtered through Celite, concentrated, and purified by CC (column chromatography) to give compound 22 as a white solid (26 mg, 0.19 mmol, 67%). ¹H NMR (600 MHz, D₂O) δ 2.77 (s, 3H), 3.06 (dd, 1H, J=4.8, 10.8 Hz), 3.22 (dd, 1H, J=4.28, 12.0 Hz), 3.36 (d, 1H, J=12.0 Hz), 3.78 (dd, 1H, J=6.6, 12.6 Hz), 3.85 (dd, 1H, J=4.8, 12.6 Hz), 3.96 (s, 1H), 4.19 (m, 1H); ¹³C NMR (150 MHz, D₂O) δ77.4, 74.9, 74.3, 61.1, 58.7, 41.3; HRMS calcd for [C₆H₁₃NO₃+H]⁺ 148.0968, found 148.0969.

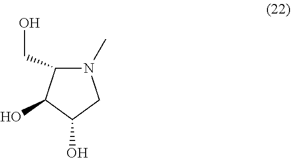

Preparation of Compound 23

A solution of compound 19 (453 mg, 1.09 mmol) was treated vinyl MgBr (3 mL, 3 mmol) in tetrahydrofuran (THF) at 0° C. After the reaction was completed, the mixture was quenched with NH₄C1, extracted with EtOAc, and concentrated. The residue was treated with Zn (500 mg, 7.6 mmol), Boc₂O (1.3 mL, 5.5 mmol) and AcOH (0.9 mL, 15.6 mmol), in DCM for 12 hours. After the reaction was completed, the mixture was filtered, quenched with 1N NaOH, extracted with DCM, concentrated, and purified. The intermediate (281 mg, 0.53 mmol) was dissolved into methanol, and O₃ gas was bubbled into the solution at −78° C. until the solution turned blue. The reaction was quenched with Me₂S and concentrated. The crude residue was dissolved into MeOH and treated with NaBH₄ (60 mg, 1.5 mmol) at 0° C. for 3 hours. After removing solvent, the reaction was extracted with water and EtOAc. The organic layers were dried with MgSO₄ and concentrated. The residue was dissolved into MeOH, and treated with palladium hydroxide in a hydrogen atmosphere for 24 hours. The reaction mixture was filtered through Celite, concentrated, and purified by CC (column chromatography) to give compound 23 as a yellowish oil (57 mg, 0.35 mmol, 32% in three steps). ¹H NMR (600 MHz, D₂O) δ 3.45-3.50 (m, 2H), 3.78 (dd, 2H, J=6.0, 12.6 Hz), 3.85 (dd, 2H, J=3.6, 12.6 Hz), 3.99-4.03 (m, 2H); ¹³C NMR (150 MHz, D₂O) δ 74.2 (×2), 62.3 (×2), 57.8 (×2); HRMS calcd for [C₆H₁₃NO₄+H]⁺ 164.0917, found 164.0918.

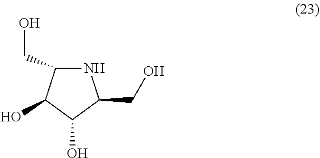 [SMILES: OC[C@@H]1N[C@@H](CO)[C@H](O)[C@H]1O]

Preparation of Compound 24

The reaction was carried out as described for compound 21 starting from cyclic nitrone 20 (150 mg, 0.36 mmol) to give compound 24 as a yellowish oil (25 mg, 0.19 mmol, 67%). ¹H NMR (600 MHz, D₂O) δ 3.29 (dd, 1H, J=2.4, 12.6 Hz), 3.51 (dd, 1H, J=4.2, 12.6 Hz), 3.54 (ddd, 1H, J=4.2, 8.4, 12.0 Hz), 3.76 (dd, 1H, J=8.4, 12.4 Hz), 3.88 (dd, 1H, J=4.2, 12.4 Hz), 4.01 (dd, 1H, J=3.6, 3.7 Hz), 4.17-4.21 (m, 1H); ¹³C NMR (150 MHz, D₂O) δ75.6, 74.2, 66.6, 58.9, 49.9; HRMS calcd for [C₅H₁₁NO₃+H]⁺ 134.0812, found 134.0814.

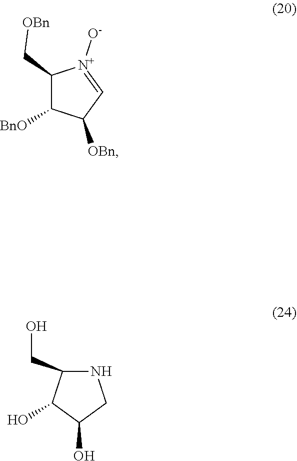 [SMILES: [O-][N+]1=C[C@@H](OCc2ccccc2)[C@H](OCc2ccccc2)[C@H]1COCc1ccccc1 ; OC[C@H]1NC[C@@H](O)[C@@H]1O]

Preparation of Compound 25

The reaction was carried out as described for compound 23 starting from cyclic nitrone 20 (513 mg, 1.23 mmol) to give compound 25 as a yellowish oil. (52 mg, 0.32 mmol, 26% in three steps). ¹H NMR (600 MHz, D₂O) δ 3.46-3.51 (m, 2H), 3.78 (dd, 2H, J=6.0, 12.0 Hz), 3.85 (dd, 2H, J=3.6, 12.0 Hz), 3.98-4.03 (m, 2H); ¹³C NMR (150 MHz, D₂O) δ 74.2 (×2), 62.3 (×2), 57.7 (×2); HRMS calcd for [C₆H₁₃NO₃+H]⁺ 164.0917, found 164.0918.

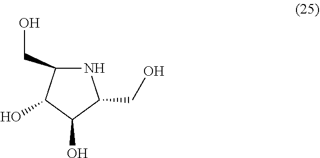 [SMILES: OC[C@H]1N[C@H](CO)[C@@H](O)[C@@H]1O]

In vitro Stabilization of Recombinant Human α-Glucosidase (rh-GAA)

rhGAA was employed in the present invention to evaluate the efficacy of specified compounds (i.e., compounds 7, 18, and 21-25) in stabilizing the activity of rhGAA. For the purpose of determining the stability of rhGAA under heat treatment, 20 μl of rhGAA (pH 7.0) was incubated in DMEM medium on ice for 10 minutes followed by heating at 48° C. for 15, 30, 45 or 60 minutes so as to heat-inactivate (denature) the rhGAA. Then, the samples were diluted with twenty-fold volume of 0.1 M citric phosphate buffer (pH 4.6), and immediately incubated with substrate (1 mM 4-methylumbelliferyl-α-D-glucoside, 4-MU-α-D-glucoside) at 37° C. for 15 minutes before quenching with glycine buffer. Liberated 4-methylumbelliferone was measured (excitation wavelength: 355 nm, emission wavelength: 460 nm). Enzyme activity was calculated relative to the unheated enzyme.

Thermal Stability Shift Assay

The stability of rhGAA was assessed using a modified fluorescence thermal stability assay on a Rotor-Gene system in DMEM or neutral pH buffer (potassium phosphate, pH 7.0). Briefly, rhGAA (2 μg) was mixed with SYPRO® Orange and various concentrations of compounds in a final reaction volume of 20 μl. A thermal gradient was applied to the plate at a rate of 1° C. per minute, during which time the fluorescence of SYPRO® Orange was continuously monitored. The fluorescence intensity at each temperature was normalized to the maximum fluorescence after complete thermal denaturation.

GAA Activity Assay in Pompe Fibroblast

Pompe fibroblosts were seeded in a sterile, clear-bottom, 48-well plate (20,000 cells per well) followed by the incubation at 37° C., 5% CO₂ for 12-16 hours. The cells were then incubated with rhGAA (0.05-5 μmol/L) with or without compounds (0-100 μmol/L) for 24 hours. After washing with growth medium for three times, the cells were incubated in growth medium at 37° C., 5% CO_{2.} Two days later, the cells were washed twice with phosphate buffered saline (PBS), and homogenized in 50 μl of citric phosphate buffer (pH 4.6) containing 0.1% TRITON™X-100 followed by centrifugation. The supernatant (20 μl) was mixed with the substrate solution (4 mM of 4-MU-α-glucoside in 0.1 M citric phosphate buffer (pH 4.6); 20 μl), and incubated at 37° C. for 1 hour. Stop solution (0.5 mol/L Na₂CO₃, pH 10.8) was then added to the mixture, and the fluorescence was read on a plate reader (excitation wavelength: 355 nm, emission wavelength: 460 nm). Raw fluorescence counts were background subtracted, as defined by counts from substrate solution only.

Determination of Inhibition Activities for Human Glycosidases

The initial velocities of hydrolysis at 37° C. were measured at 100 mM sodium phosphate buffer (pH 4.5) with 4-MU-glycopyranoside at 355 nm excitation and 460 nm emission using multi-detection reader. The assay was performed in a 96-well microtiter plate.

Cytotoxicity

Normal fibroblasts were seeded in a 96-well plate at a number of 5,000 cells per well. 24 hours later, the medium were renewed, and specified compounds were respectively added to the cells at a final concentration of 10-200 μM. All compounds were dissolved in DMSO or H₂O, and control experiments were performed with DMSO. Cells were incubated at 37° C. in 5% CO₂ for 48-72 hours. Then, 10 μl of ALAMARBLUE®was added to the cells followed by incubation at 37° C. in 5% CO₂ for additional 3-5 hours. The number of viable cells was quantified and measured at an excitation wavelength of 560 nm, and an emission wavelength of 590 nm.

Animal Model

Three months old male Pompe mice (B6;129-GaatmlRabn/J) were used for the experiments.

The Pompe mice (n=3-4 each group) were treated with co-administration of rhGAA (40 mg/kg) and small molecules (100 mg/kg or 200 mg/kg of NB-DNJ; or 100 mg/kg or 200 mg/kg of compound 21) at single dose via tail vein injection. Mice then were sacrificed 72 hours after injection, and GAA activity of heart was measured. Wild-type mice, untreated Pompe mice, and the mice merely injected with rhGAA (40 mg/kg) were used as control groups. For short-term glycogen clearance study, Pompe mice were treated with intravenous rhGAA (20 mg/kg), with oral NB-DNJ (10 mg/kg) or compound 21 (10 mg/kg) weekly, and compared with untreated Pompe mice for 3 weeks. Mice also received methotrexate intraperitoneally within 15 minutes, 24 hours and 48 hours after the first rhGAA therapy; and diphenhydramine was injected intraperitoneally 10 minutes before each rhGAA therapy to reduce the immune response of mice. After 3 weeks, the mice were sacrificed and Glycogen content of heart was analyzed.

### Example 1

### Evaluation of ADMDP Stereoisomers to Stabilize rhGAA

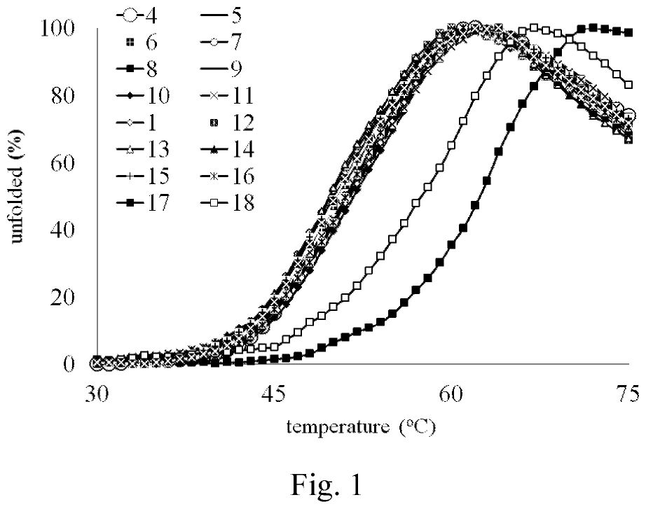

For the purpose of evaluating the ability to stabilize rhGAA, different ADMDP stereoisomers were individually incubated with rhGAA under neutral environment (pH 7.0) at 4° C. for 10 minutes. The enzyme melting temperature (Tm) was then measured by fluorescence-based thermal denaturation assay. As the data of FIG. 1 depicted, in the absence of compounds, the Tm of rhGAA was 50.2° C.; compared with other ADMDP stereoisomers that did not obviously alter (e.g., increase or decrease) the Tm value of rhGAA, the administration of 1 mM of compounds 17 and 18 respectively increased the Tm of rhGAA from 50.2° C. to 62.4° C. and 57.5° C. The results demonstrated that both compounds 17 and 18 improved the stability of rh-α-glu under physiological condition (pH 7.0) (FIG. 1). Structurally, these two compounds share the same configuration pattern (3S,4S,5S), and the only difference is the chiral center at the C2 positon. This finding indicates that this configuration pattern should play a critical role for the recognition and stabilization of rh-α-glu.

### Example 2

### Evaluation of ADMDP Derivatives to Stabilize rhGAA

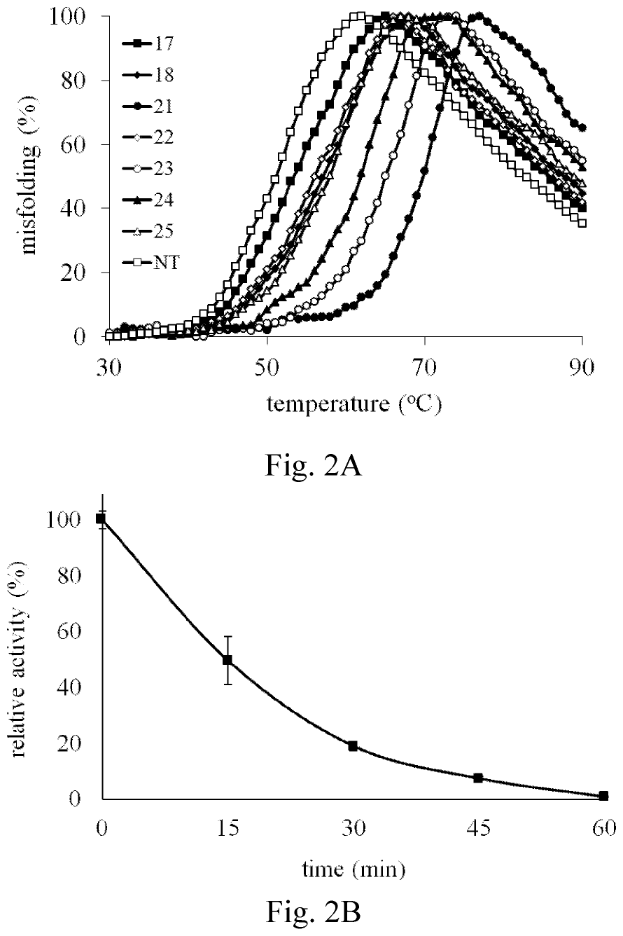

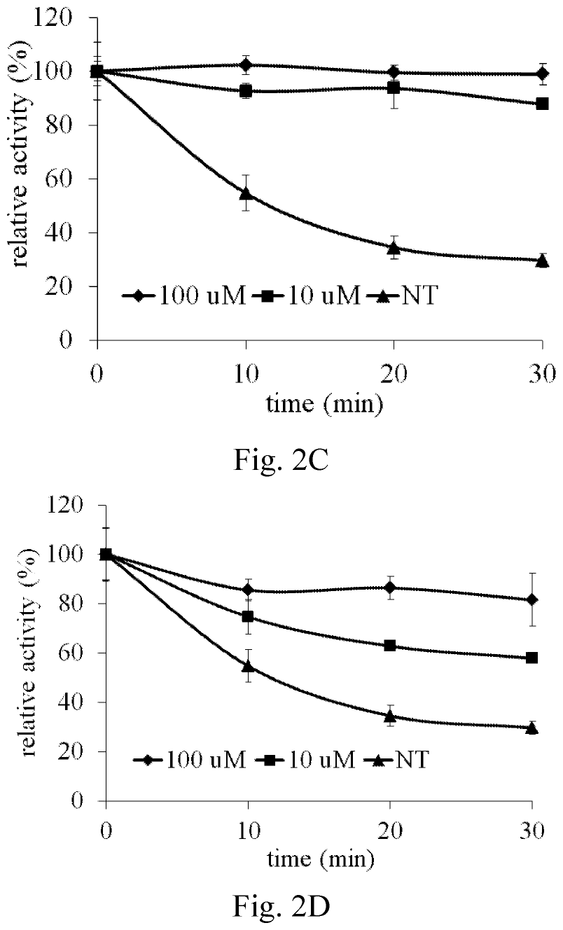

In this example, two ADMDP stereoisomer (i.e., compounds 17 and 18) and five ADMDP derivatives (i.e., compounds 21-25) were respectively incubated with rhGAA so as to evaluate their stabilizing effect on rhGAA. After incubating with 100 μM of specified compound, the Tm value of rhGAA was measured by thermal shift assay. The results were respectively depicted in FIGS. 2A-2D.

The data of 2A indicated that among tested compounds, compounds 21 and 23 exhibited the highest effect on stabilizing rhGAA, in which the Tm of rhGAA incubated with compounds 21 and 23 raised from 50.2° C. to 69.8° C. and 65° C., respectively. In contrast, the D-form analogues, including compounds 24 and 25, were not as potent as the L-forms 21 and 23 (FIG. 2A). Importantly, the shifted Tm was significantly reduced when the endocyclic amine on 21 was methylated (FIG. 2A). This finding indicated that the amino group plays an important role for the hydrophilic interaction with rhGAA.

Next, the ability of potential compounds 21 and 23 in protecting the enzyme from heat-induced denaturalization was also evaluated. As the data of FIGS. 2B and 2C depicted, the activity of rhGAA, which was incubated in culture medium (i.e., DMEM medium; FIG. 2B) or phosphate buffer (pH 7.0; FIG. 2C), gradually decreased as the heating time increased. The administration of compound 21 or 23 obviously improved the enzyme activity (FIGS. 2C and 2D). It is further noted that compound 21 suppressed the inactivation of enzyme at both 100 and 10 μM (residue activity>90%), while the enzyme activity was dropped to 80% and 60% when incubated with 100 and 10 μM of compound 23, respectively (FIGS. 2C and 2D). To further test the stabilizing activity of compound 21, rhGAA was incubated with compound 21, or N-butyl-deoxynojirimycin (NB-DNJ), a potent rhGAA stabilizer, in DMEM at 37° C. We found that compound 21 was capable of maintaining enzyme activity at about 60%, while NBDNJ maintained enzyme activity at about 30% after 40 minutes of incubation (data not shown). Such experiments suggested that compound 21 can stabilize rhGAA and maintain the activity of rhGAA in culture medium (i.e., DMEM), which potentially prevents the enzyme from inactivation before being uptaken by cells.

The results suggested that compound 21 may serve a potential lead compound for the treatment of diseases caused by or associated with GAA deficiency or malfunction, for example, Pompe disease.

### Example 3

### Evaluating Cellular Effect of ADMDP Derivatives

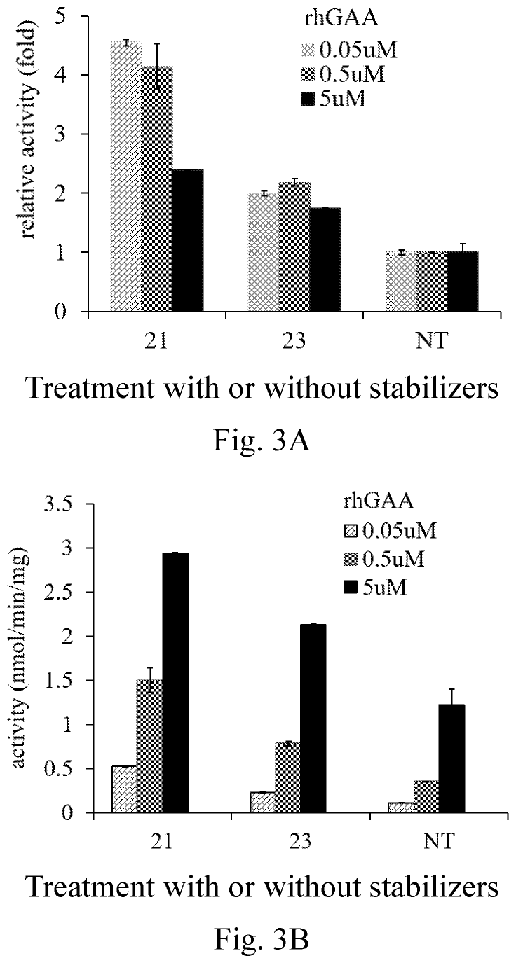

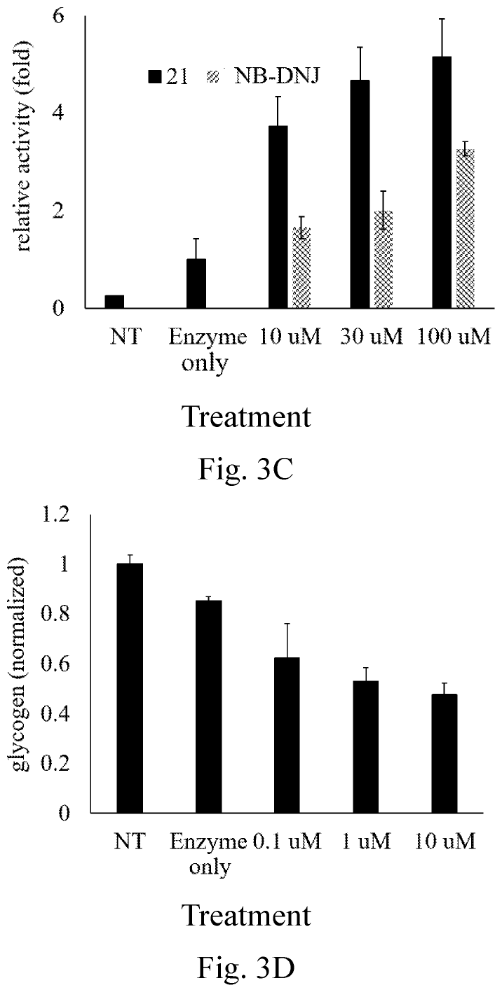

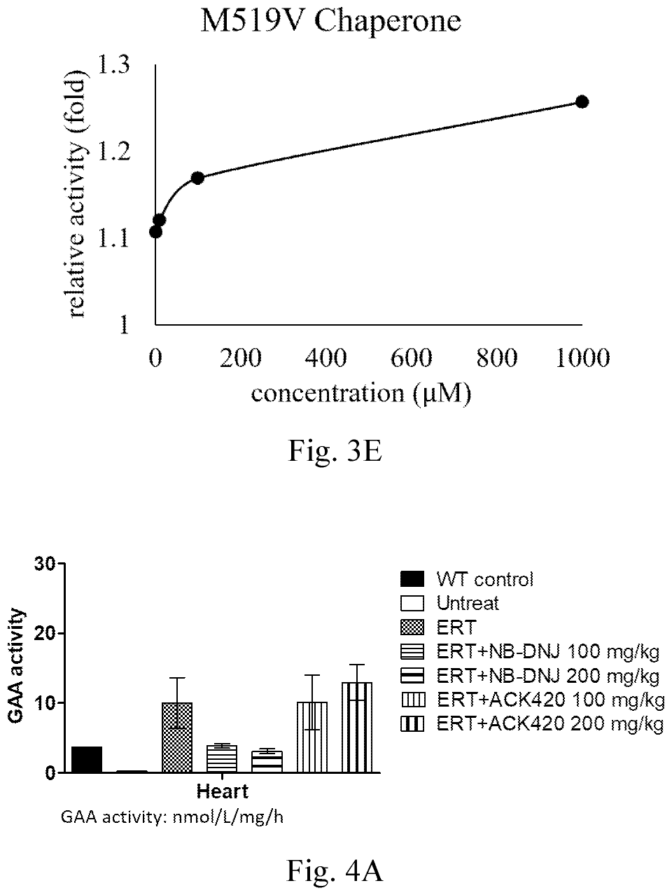

These promising results of 21 and 23 to prevent rhGAA from protein denaturalization or deactivation prompted us to further investigate the effect of 21 and 23 on D645E or M519V cells, two cell lines derived from Pompe fibroblasts. NB-DNJ served as a positive control in this experiment. The results were respectively depicted in FIGS. 3A-3E.

As the data summarized in Table 2, neither compound 21 nor compound 23 induced a cytotoxic response in cells.

TABLE 2
Cytotoxicity of compounds 21 and 23 toward fibroblasts

| Compound ID | Cytotoxicity |
|---|---|
| 21 | N.D. |
| 23 | N.D. |

N.D.: not detected

Different concentrations (i.e., 0.05, 0.5, or 5 μM) of rhGAA were added to D645E cells in the presence or absence of specified compound (50 μM). The activity of GAA in D645E cells was measured three days post-treatment. The data of FIG. 3A indicated that compared to the control group (i.e., NT group), both compounds 21 and 23 improved the activity of rhGAA. As depicted in FIG. 3B, the GAA activities were 0.1, 0.4, and 1.2 nmol/min/mg in the cells respectively treated with 0.05, 0.5, and 5 μM of rhGAA (see, the “NT” group in FIG. 3B), and the co-administration of compound 21 or 23 with rhGAA obviously improved the GAA activity in cells (see, the “21” and “23” groups in FIG. 3B). Notably, the co-administration of 21 enhanced intracellular rhGAA activity by 2-to 4.5-fold (FIG. 3A-3C). The data of FIG. 3C further demonstrated that compound 21 (0-100 μM) improved the activity of rhGAA (0.05 μM) in a dose-dependent manner, in which co-administration of 10, 30, and 100 μM of compound 21 respectively increased the intracellular GAA activity by 3.7-, 4.9- and 5.6-fold. The maximal increase of the enzyme activity measured at 100 μM of compound 21 reached 5.6-fold, as compared to the Mock treatment (i.e., the cells treated with 0.05 μM of rhGAA without compound 21 added; the “Enzyme” group in FIG. 3C). Surprisingly, the stabilizing effect of compound 21 on rhGAA was significantly better than that of NB-DNJ (3.4-fold) (FIG. 3C). Since Pompe disease is caused by a deficiency of GAA that leads to the accumulation of glycogen in affected cells, the effect of compound 21 on the level of glycogen was also examined in D645E fibroblast. As the data depicted in FIG. 3D, compared to treatment of 0.1 uM rhGAA (“Enzyme”), which moderately reduced cellular glycogen content (15%), the treatment of 0.1, 1, or 10 μM of compound 21 obviously reduced the level of glycogen (about 50%; “NT” vs rhGAA in the presence of 10 uM of compound 21) in patient cells. The data of FIG. 3D demonstrated that enzyme stabilizer 21 was able to improve the clearance of glycogen in patient cells.

To test the stabilizing activity of 21 toward mutant GAA, compound 21 was added to M519V fibroblasts, another fibroblasts isolated from Pompe cells, and the intracellular GAA activity was determined four days post-treatment. The data of FIG. 3E indicated that compound 21 dose-dependently increased intracellular GAA activity in M519V fibroblasts. Cytotoxicity of 21 and 23 was also detected, and there was no observable effect even treating at 1,000 μM toward normal fibroblast (Data not shown).

### Example 4

### In Vivo Study

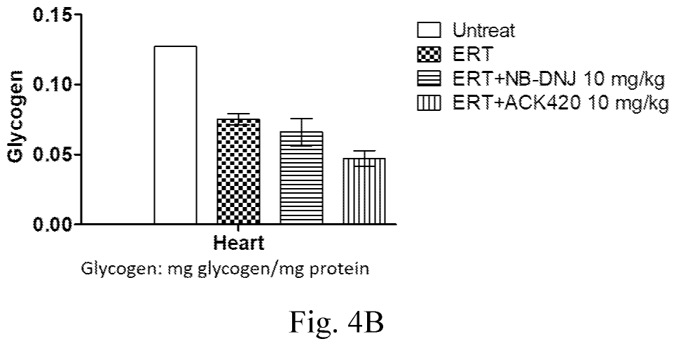

In this example, the therapeutic effect of compound 21 on Pompe disease was evaluated by a mouse model. The results were respectively depicted in FIGS. 4A and 4B.

Compared to the untreated control, the administration of rhGAA (i.e., the “ERT” group) increased GAA activity (FIG. 4A) and decreased glycogen content (FIG. 4B) in the heart of Pompe mice. It was noted that the GAA activity of mice treated with the combination therapy of ERT and compound 21 (i.e., the “ERT+compound 21” group) was obviously higher than that of mice treated with the combination treatment of ERT and NB-DNJ (i.e., the “ERT+NB-DNJ” group) (FIG. 4A), while the mice treated with the combination therapy of ERT and compound 21 had lower glycogen content in the heart thereof as compared to the mice treated with the combination therapy of ERT and NB-DNJ (FIG. 4B). The data of FIGS. 4A and 4B demonstrated that compound 21 of the present invention was useful in improving the activity of rhGAA and decreasing the level of glycogen accumulated in the subject having Pompe disease, and accordingly provides a potential means to treat Pompe disease.

In conclusion, the present disclosure demonstrated that certain ADMDP stereoisomers and their derivatives, including compounds 17, 18, and 21-25, are useful in stabilizing the activity of rhGAA. Based the results, each of the specified compounds (i.e., compounds 17, 18, and 21-25) may be employed as a stabilizer of rhGAA thereby enhancing the therapeutic effect of rhGAA on treating α-glucosidase-associated diseases, such as Pompe disease.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

1-16. (canceled)

17. A method of treating Pompe disease in a subject, comprising administering to the subject a first therapeutic effective amount of a compound of formula (I), or a salt, an ester or a solvate thereof, 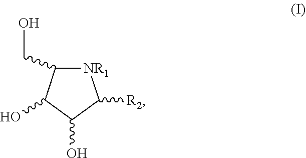
wherein R₁ and R₂ are independently H or alkyl optionally substituted by —NH₂ or —OH.

18. The method of claim 17, wherein
R₁ is H, and R₂ is H or methyl optionally substituted by —NH₂ or —OH; or
R₁ is methyl, and R₂ is H.

19. The method of claim 18, wherein the compound of formula (I) is selected from the group consisting of,

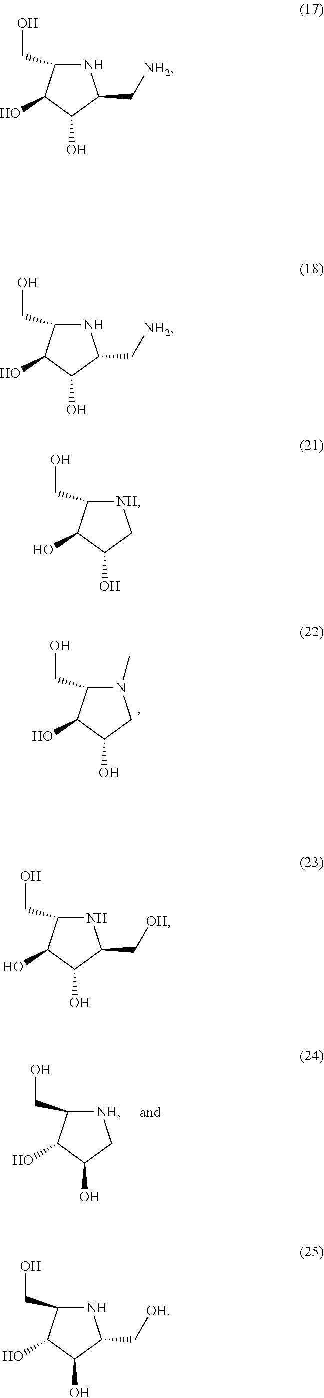

20. The method of claim 19, wherein the compound of formula (I) is selected from the group consisting of,

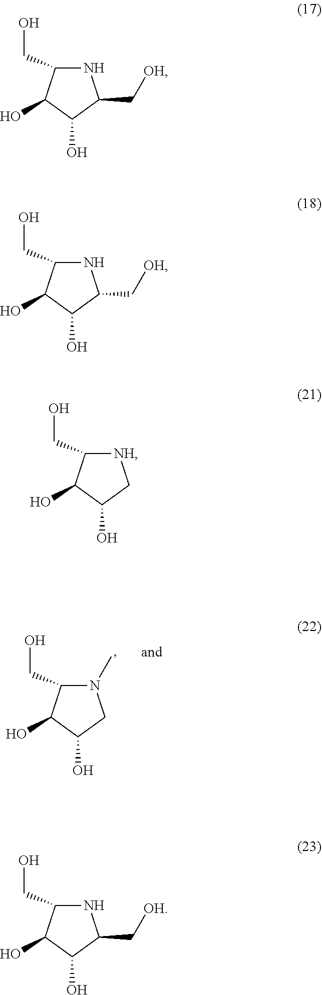

21. The method of claim 20, wherein the compound of formula (I) is

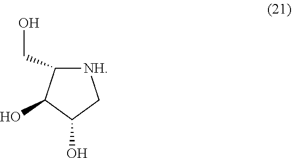

22. The method of claim 20, wherein the compound of formula (I) is

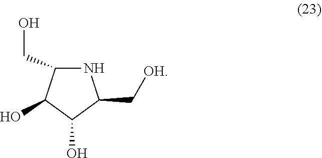 [SMILES: OC[C@@H]1N[C@@H](CO)[C@H](O)[C@H]1O]

23. The method of claim 17, further comprising administering to the subject a second therapeutically effective amount of an α-glucosidase, prior to, concurrently with, or after the administration of the compound of formula (I), or the salt, the ester or the solvate thereof.

24. The method of claim 17, wherein the first therapeutic effective amount is about 0.01 mg/Kg to 10 g/Kg.

25. The method of claim 17, wherein the subject is a human.
